(19) United States
(12) Patent Application Publication
(10) Pub. No.: US 2024/0212862 A1
(43) Pub. Date: Jun. 27, 2024

(54) GENERAL MULTI-DISEASE PREDICTION SYSTEM BASED ON CAUSAL CHECK DATA GENERATION

(71) Applicant: ZHEJIANG LAB, Hangzhou (CN)

(72) Inventors: Jingsong LI, Hangzhou (CN); Feng WANG, Hangzhou (CN); Hang ZHANG, Hangzhou (CN); Shengqiang CHI, Hangzhou (CN); Yu TIAN, Hangzhou (CN); Tianshu ZHOU, Hangzhou (CN)

(21) Appl. No.: 18/595,379

(22) Filed: Mar. 4, 2024

Related U.S. Application Data
(63) Continuation of application No. PCT/CN2023/089993, filed on Apr. 23, 2023.

(30) Foreign Application Priority Data
May 20, 2022 (CN) 202210547826.4

(51) Int. Cl. G16H 50/50; G06N 3/0475; G06N 5/022

(52) U.S. Cl. CPC G16H 50/50; G06N 3/0475; G06N 5/022

(57) ABSTRACT

Disclosed is a general multi-disease prediction system based on causal check data generation. For a general scenario, the present invention provides a tendency score calculation method based on a general tendency score network from the perspective of causality; compared with the problem of poor interpretability of traditional generative adversarial networks, the present invention provides a generative adversarial network based on causal check, so that generated data better conforms to real causal logic; in view of the problem that existing graph convolutional neural networks are modeled only from the perspective of correlation, the present invention provides a general multi-disease prediction model based on a general causal graph convolutional neural network, and a causal effect value is integrated to improve the prediction performance of the general multi-disease prediction system on diseases, thereby solving the problems of poor model performance and low robustness caused by few training samples in a general scenario.

### CROSS-REFERENCE TO RELATED APPLICATIONS

The present application is a continuation of International Application No. PCT/CN2023/089993, filed on Apr. 23, 2023, which claims priority to Chinese Application No. 202210347826.4, filed on May 20, 2022, the contents of both of which are incorporated herein by reference in their entireties.

### TECHNICAL FIELD

The present application belongs to the technical field of medical and health information and, in particular, to a general multi-disease prediction system based on causal check data generation.

### BACKGROUND

With the development of information technology, machine learning has become an important force to promote medical development. General medicine, as the most widely accepted medical discipline in the medical field, is one of the key areas in which a machine learning model is applied in medical scenes. However, due to the complexity of general diseases and the high cost of sample acquisition, it is difficult for some diseases to obtain a large number of training data, which leads to the poor prediction effect of the existing general multi-disease prediction system for few-shot diseases. At present, a set of general multi-disease prediction systems for few-shot diseases is urgently desired.

Generating simulation data by a data generation method is a common method to solve the problem of insufficient training samples of a machine learning model. The existing data generation methods are mainly based on generative adversarial networks. The generative adversarial network performs well in generating image data. However, in the general practice scene, there are many kinds of data and complex structures, especially the structured medical data, which contains many kinds of patient-centered characteristic data, with heterogeneity in time and space, and the data distribution is more complicated. It is difficult for the traditional generative adversarial network to handle the structured data with complex distribution. On the one hand, training with few sample data is prone to the problems such as unstable training, gradient disappearance or pattern collapse. On the other hand, only considering the correlation between variables, without considering the causal relationship between variables, leads to the result that the generated data is difficult to understand and inconsistent with common sense. Using these data for model training may not improve or even weaken the training effect of the model. For example, colds include viral colds and bacterial colds, and thus two kinds of drugs should be used, respectively. If the data of fever patients are generated based on a correlation model, it may result in the simultaneous use of viral cold medicines and bacterial cold medicines, which will interfere with the subsequent model construction.

The method for calculating causal effect values based on propensity scores is the most common method to measure the causal relationship between variables. Most of the existing methods for calculating propensity scores are based on logistic regression. However, due to the variety, complex structure and linear inseparability of data in the general practice scenes, the method for calculating propensity scores based on logistic regression does not perform well in general practice scenes.

### SUMMARY

In view of the shortcomings of the prior art, the present application provides a propensity score calculation method based on a general propensity score network from the perspective of causality, and on this basis, provides a medical data generation method based on causal check, which solves the problem that the data generated by a general propensity score network based on correlation analysis is difficult to understand, constructs a general multi-disease prediction system, and solves the problems of poor model performance and low robustness caused by few training samples in the general practice scenes.

The object of the present application is achieved through the following technical solution: a general multi-disease prediction system based on causal check data generation, including:
(1) a disease statistics module which is configured to count a sample number of various general diseases, and obtain few-shot general diseases according to a sample ratio of various general diseases; where the sample ratio is a ratio of a sample number of diseases with a largest number of samples to the sample number of various general diseases, and for a general disease with a sample ratio greater than a set threshold, the general disease is added into a few-shot general disease set R, and a frequency of a r^{th} few-shot general disease is calculated, qᵣ=countᵣ/Σ_{r∈R} countᵣ, where countᵣ is a sample number of the r^{th} few-shot general Er_{E}R countᵣ disease;
(2) a causal check module which is configured to construct a first event variable set according to a characteristic variable set of general patients and construct a second event variable set according to a disease label variable set of general patients, where any first event variable and any second event variable form an event pair;
the causal check module is further configured to construct and train a general propensity score network and calculate a general propensity score by using the trained general propensity score network, where a general propensity score represents a probability that a first event occurs to the general patient under a covariant condition; and causal effect values of all event pairs are calculated by using the general propensity score;
(3) a data generation module configured to construct a data generation model for the few-shot general diseases based on a generative adversarial network of causal check, and generate simulated data by using the trained data generation model;
where the data generation model includes a generator and a discriminator, and the generator and the discriminator are trained iteratively and alternately;
a training process of the generator includes: generating a random noise for each few-shot general disease, and inputting the random noise and a corresponding disease label into the generator to obtain a generated sample; calculating the causal effect values of all event pairs of the generated sample; inputting the generated sample and the corresponding disease label into the discriminator to obtain a discrimination result; where a total loss of the generator includes a discriminator adversarial loss, a causal loss and a regularization term loss; the causal loss is a KL divergence loss between the causal effect values of all event pairs of the generated sample, corrected by the frequency of the few-shot general disease, and the causal effect values of all event pairs of original data;
a training process of the discriminator includes: randomly extracting positive samples from the original data, and extracting negative samples with a same number with the positive samples but with different disease labels; generating a same number of random noises, and using the generator to obtain the generated sample; inputting the positive samples, the negative samples and the generated samples into the discriminator, respectively, to obtain the discrimination result; and
(4) a model prediction module which is configured to obtain characteristic data and disease label data of a general patient to be trained, and generate general disease data by using the data generation model for the few-shot general diseases; where training samples and generated general disease data are jointly used to train a general multi-disease prediction model based on a general causal graph convolutional neural network, and the trained general multi-disease prediction model is used to predict general diseases.

Further, in the causal check module, the general propensity score network is trained by using binary variable data of the general patient; characteristic variable data and label variable data of the general patient are converted into binary variables, categorical variables are converted into the binary variables by One-Hot Encoding, and continuous variables are converted into the categorical variables by binning in advance, and the categorical variables are further converted into the binary variables by One-Hot Encoding.

Further, the general propensity score network includes an input layer, a locally-connected layer, a sigmoid activation layer and an output layer;
a number of codes in the input layer and a number of codes in the output layer are both a number M of first event variables in the first event variable set; both the locally-connected layer and the sigmoid activation layer contain τM nodes, τ≥2; a u^{th} node of the input layer is connected with all nodes except those from a τ(u−1)+1^{th} to a τu^{th} node in the locally-connected layer; the nodes from the τ(u−1)+1^{th} to the τu^{th} node in the locally-connected layer are connected with nodes from a τ(u−1)+1^{th} to a τu^{th} node in the sigmoid activation layer in one-to-one correspondence; the nods from the τ(u−1)+1^{th} to the τu^{th} node in the sigmoid activation layer are only connected with a u^{th} node in the output layer.

Further, the training process of the general propensity score network is as follows:
for each first event variable a, covariant data corresponding to the training samples is input into the locally-connected layer to obtain a first characteristic representation of propensity; the first characteristic representation of propensity is input into the sigmoid activation layer to obtain a second characteristic representation of propensity; the second characteristic representation of propensity is input into the output layer to obtain a predicted value of the first event variable a; a propensity loss is calculated by using the predicted values of all the first event variables and true values of all the first event variables.

Further, in the causal check module, the trained general propensity score network is used to calculate a general propensity score pᵢ^{a} of a general patient i for a first event variable a, and a causal effect value ATE_{a,b} of the first event variable a and a second event variable b are calculated by using the general propensity score according to the following calculation formula:

$${ATE}_{a , b} = \frac{1}{n} {\sum }_{i = 1}^{n} \frac{{T}_{i} {Y}_{i}}{{p}_{i}^{a}} - \frac{1}{n} {\sum }_{i = 1}^{n} \frac{( 1 - {T}_{i} ) {Y}_{i}}{1 - {p}_{i}^{a}}$$

where n represents a total number of patients to be studied, Tⱼ represents a true value of the first event variable of an i^{th} patient, and Yᵢ represents a true value of the second event variable of the i^{th} patient.

Further, in the data generation module, the generator is composed of multiple layers of generator modules; the generator module includes a normalization layer, a fully-connected layer and an activation layer; the activation layer of the generator module in a last layer is a sigmoid activation layer; in a training process, the random noises and the corresponding disease labels are input into the normalization layer of a first generator module, and normalized data are input into the fully connected layer of the first generator module to obtain a first characteristic representation of input data; the first characteristic representation is input into the activation layer of the first generator module to obtain a second characteristic representation of the input data, and the second characteristic representation is used as input data of the generator module in a next layer; and finally the generated sample is obtained through the sigmoid activation layer of the generator module in the last layer.

Further, in the data generation module, a formula for calculating a causal loss L_{causal} is as follows:

$${L}_{casual} = {\sum }_{r ∈ R} {q}_{r} {\sum }_{a ∈ {A}_{r}} ( {ATE}_{a , r}^{g} log ( {ATE}_{a , r}^{g} ) - {ATE}_{a , r}^{g} log ( {ATE}_{a , r}^{o} ) )$$

where, ATE_{a,r}^{o} represents a causal effect value of a first event variable a and a second event variable r of the original data, and ATE_{a,r}^{g} represents a causal effect value of the first event variable a and the second event variable r of the generated sample; Aᵣ represents the first event variable set paired with the second event variable r; the second event variable set is a general disease set, and the second event variable r corresponds to a few-shot general disease r in the few-shot general disease set R.

Further, in the data generation module, a formula for calculating a discriminator adversarial loss L_{ζ} is as follows:

$${L}_{ζ} = \frac{1}{N} {\sum }_{i = 1}^{N} - log ( {y}_{i}^{*} )$$

where N is a data size of the random noises, and yᵢ* is a probability that an i^{th} generated sample is determined as real data of a corresponding disease by the discriminator;

a calculation formula of a regularization term loss L_{regular} is as follows:

$${L}_{regular} = ‖ w ‖$$

where, ∥·∥ represents a L1 norm and w represents a model parameter of the generator.

Further, in the data generation module, a formula for calculating a total loss L_{d} of the discriminator is as follows:

$${L}_{d} = - \frac{1}{{m}_{d}} {\sum }_{k = 1}^{{m}_{d}} ( log ( D ( {x}_{k} , {y}_{k} ) ) + log ( 1 - D ( {x}_{k}^{∧} , {y}_{k} ) ) + log ( 1 - D ( {d}_{k} , {y}_{k} ) ) )$$

where is m_{d} is a number of the positive samples, yₖ is a disease label corresponding to the positive samples, and xₖ, xₖ\hat{ }, dₖ are k^{th} extracted positive sample, k^{th} extracted negative sample and k^{th} generated sample obtained by using the generator, respectively, and D(xₖ, yₖ), D(xₖ\hat{ }, yₖ), D(dₖ,yₖ) are probabilities that the positive sample xₖ, the negative sample xₖ\hat{ } and the generated sample dₖ are determined as the real data of a disease yₖ by the discriminator.

Further, the model prediction module is configured to:

construct an event relation graph: each first event variable constitutes a first event node in the event relation graph, each second event variable constitutes a second event node in the event relation graph, and an edge is constructed for each event pair;

generate a node embedding representations of the first event node and a node embedding representation of the second event node, construct a degree matrix Φ and an adjacency matrix A based on the event relation graph, and construct a causal effect matrix Ψ using the causal effect values of the original data;

construct the general multi-disease prediction model based on the general causal graph convolutional neural network, where the general causal graph convolutional neural network includes a plurality of causal graph convolutional modules, and each of the causal graph convolutional module includes a causal graph convolutional layer and an activation layer;

input the node embedding representations into the causal graph convolutional layer of a first causal graph convolutional module to obtain a first graph characteristic representation h⁽⁰⁾:

$${h}^{( 0 )} = {{Φ}^{- \frac{1}{2}} ( ( A + I ) * Ψ )}^{- \frac{1}{2}} {H}^{( 0 )} {W}^{( 0 )}$$

where, H⁽⁰⁾ represents a node embedding representation, W⁽⁰⁾ represents a weight of the causal graph convolutional layer, I represents an identity matrix, and * represents a multiplication of elements of the matrix;

input h⁽⁰⁾ into the activation layer of the first causal graph convolutional module to obtain an output H⁽¹⁾ of the first causal graph convolutional module; and

input the output of the previous causal graph convolutional module into a next causal graph convolutional module until a final disease prediction result is obtained.

The present application has the following beneficial effects:
1. In the present application, the causal logic between characteristics is considered while data is amplified, so that the generated data is more in line with the real situation, and model training of this part of data can improve the model performance.
2. Compared with the problem of poor interpretability of the traditional generative adversarial network, the present application proposes a generative adversarial network based on causal check, which makes the generated data more consistent with the real causal logic and has certain causal interpretability.
3. Aiming at the problem that the existing graph convolutional neural network only models from the perspective of correlation, the present application proposes a general causal graph convolutional neural network to improve the robustness of the general multi-disease prediction model.

### BRIEF DESCRIPTION OF DRAWINGS

FIG. 1 is a structural block diagram of a general multi-disease prediction system based on causal check data generation provided by an embodiment of the present application;

FIG. 2 is a flowchart of the implementation of the causal check module provided by an embodiment of the present application;

FIG. 3 is a structural diagram of a general propensity score network provided by an embodiment of the present application;

FIG. 4 is a structural diagram of a generative adversarial network based on causal check provided by an embodiment of the present application; and

FIG. 5 is a flowchart of the implementation of the model prediction module provided by an embodiment of the present application.

### DESCRIPTION OF EMBODIMENTS

In order to better understand the technical solution of the application, the embodiments of the application will be described in detail with the attached drawings.

It should be clear that the described embodiments are only part of the embodiments of this application, not all of the embodiments. Based on the embodiments in this application, all other embodiments obtained by those skilled in the art without creative work belong to the protection scope of this application.

The terms used in the examples of this application are intended to describe specific embodiments only and are not intended to limit this application. The singular forms “a”, “said” and “the” used in the embodiments of this application and the appended claims are also intended to include the plural forms, unless the context clearly indicates other meaning.

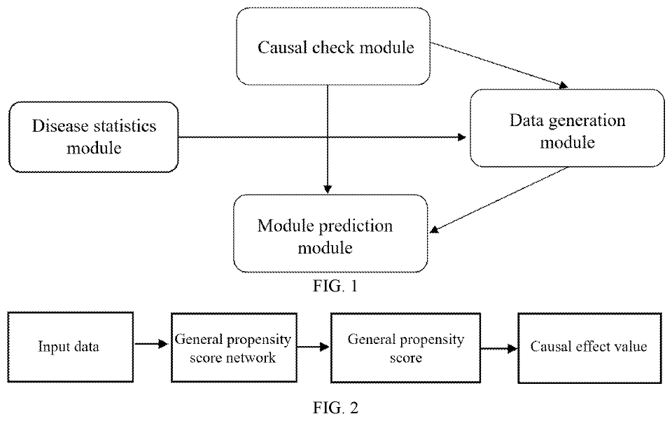

The present application provides a method for generating medical data based on a generative adversarial network of causal check, and based on this method, a set of general multi-disease prediction systems is constructed to solve the problem that the model has poor prediction for few-shot diseases due to less training samples in the general multi-disease prediction model. As shown in FIG. 1, the general multi-disease prediction system based on causal check data generation provided by the present application includes a disease statistics module, a causal check module, a data generation module and a model prediction module.

The following description further gives some examples of the implementation of each module of the general multi-disease prediction system based on causal check data generation that meets the requirements of this application.

### I. Disease Statistics Module

For all kinds of general diseases, the sample numbers of various diseases are counted and the sample ratios of various diseases are calculated. The sample ratio is the ratio of the number of samples of the diseases with the largest number of samples to the number of samples of various diseases. For example, for the four general diseases of common cold, gastritis, diarrhea and fever, the sample numbers are 10, 20, 30 and 40 respectively, and the sample ratios are 4, 2, 4/3 and 1 respectively.

For diseases whose disease sample ratios are greater than a set threshold (which is an adjustable parameter set according to the actual situation), they are added into the few-shot general disease set R, and the frequency

$${q}_{tr} = \frac{{count}_{r}}{{Σ}_{r ∈ R} {count}_{r}}$$

of the r^{th} few-shot general disease si calculated, where countᵣ is the number of samples of the r^{th} f disease.
II. Causal Check Module, the Implementation Process of which is Shown in FIG. 2.

The patient's characteristic variable data and label variable data are obtained. The characteristic variable data and label variable data are converted into binary variables as follows. For categorical variables, they are converted into binary variables by One-Hot Encoding. For continuous variables, they are converted into categorical variables through binning, and then into binary variables through One-Hot Encoding.

The characteristic variable set constitutes a first event variable set, and the label variable set constitutes a second event variable set. The first event variable set is a set of clinical manifestations, such as {hypertension, fever, chest tightness}, and the second event variable set is a set of general diseases, such as {cold, gastritis, cardiovascular diseases}.

Any first event variable in the first event variable set and any second event variable in the second event variable set form an event pair, and the causal effect values of all event pairs are calculated. The calculation method of causal effect values is as follows.

It is recorded that a first event variable a and a second event variable b form an event pairing δ; a covariant corresponding to the event pair δ is defined as the variable except the first event variable a in the first event variable set. Taking the event pair hypertension-cold as an example, the covariant is a variable except the hypertension variable in the first event variable set {hypertension, fever, chest tightness}, that is {fever, chest tightness}. Because of the variety and complexity of the data in general scene, the traditional method of calculating the propensity score based on logistic regression has limited ability in dealing with nonlinear separable data. Therefore, the present application constructs a general propensity score network aiming at the general scene, trains the general propensity score network by using the binary variable data of general patients, and calculates the general propensity score by using the trained general propensity score network.

The general propensity score indicates the probability that the first event occurs to the patient under the covariant condition. Taking {hypertension, fever, chest tightness} as an example, it is the probability of hypertension in patients with fever and chest tightness.

The general propensity score network includes an input layer, a locally-connected layer, a sigmoid activation layer and an output layer.

Specifically, the number of codes in the input layer and a number of codes in the output layer are both a number M of first event variables in the first event variable set. Both the locally-connected layer and the sigmoid activation layer contain τM nodes, where τ is an adjustable parameter, τ≥. The u^{th} node of the input layer is connected with all nodes except those from a τ(u−1)+1^{th} to a τu^{th} node in the locally-connected layer. The nodes from the τ(u−1)+1^{th} to the τu^{th} node in the locally-connected layer are connected with nodes from a τ(u−1)+1^{th} to a τu^{th} node in the sigmoid activation layer in one-to-one correspondence. The nods from the τ(u−1)+1^{th} to the τu^{th} node in the sigmoid activation layer are only connected with a u^{th} node in the output layer. The locally-connected layer has the advantages that the locally-connected layer ensures the local connection between the input layer and the output layer; for each first event variable to be predicted, the covariant characteristic node of the input layer forms a local network with the first event variable nodes of the locally-connected layer, the sigmoid activation layer and the output layer; and the locally-connected layer ensures the mutual independence among the local networks, so that the predicted first event variable will not be used for prediction.

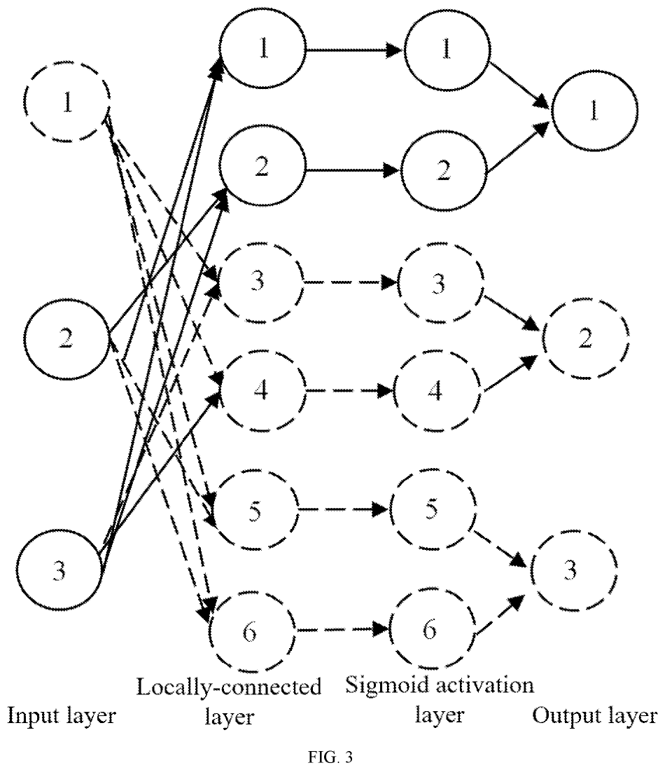

FIG. 3 is an example of a general propensity score network. In this example, M=3, τ=2, and for the input layer node 1, it is connected with all nodes in the locally-connected layer except the nodes 1 and 2; the locally-connected layer node 1 is connected with the node 1 of the sigmoid activation layer, the locally-connected layer node 2 is connected with the node 2 of the sigmoid activation layer, and the locally-connected layer nodes 1 and 2 are only connected with the node 1 of the output layer.

The training process of the general propensity score network is as follows:

for each first event variable a, covariant data corresponding to the training samples is input into the locally-connected layer to obtain a first characteristic representation of propensity; the first characteristic representation of propensity is input into the sigmoid activation layer to obtain a second characteristic representation of propensity; the second characteristic representation of propensity is input into the output layer to obtain a predicted value of the first event variable a; a propensity loss is calculated by using the predicted values of all the first event variables and true values of all the first event variables. The propensity loss Lₚ is calculated as follows

$${L}_{p} = {\sum }_{f = 1}^{{m}_{p}} {\sum }_{a = 1}^{M} ( {γ}_{f , a} log ( {γ}_{f , a}^{\#} ) - ( 1 - {γ}_{f , a} ) log ( 1 - {γ}_{f , a}^{\#} ) )$$

where mₚ represents the total number of training samples, γ_{f,a} represents the true value of the first event variable a of a training sample f, and γ_{f,a}^{#} represents the predicted value of the first event variable a of the training sample f.

The trained general propensity score network is used to calculate a general propensity score pᵢ^{a} of a general patient i for the first event variable a, and a causal effect value ATE of the first event variable and a second event variable are calculated by using the general propensity score. The formula of the causal effect value ATE_{a,b} of the first event variable a and the second event variable b is as follows:

$${ATE}_{a , b} = \frac{1}{n} {Σ}_{i = 1}^{N} \frac{{T}_{i} {Y}_{i}}{{p}_{i}^{a}} - \frac{1}{N} {Σ}_{i = 1}^{n} \frac{( 1 - {T}_{i} ) {Y}_{i}}{1 - {p}_{i}^{a}}$$

where n represents a total number of patient to be studied, and Tᵢ represents the true value of the first event variable of an i^{th} patient; Yᵢ represents the true value of the second event variable of the i^{th} patient; Yᵢ=1 represents that a second event occurs to the i^{th} patient, and Yᵢ=0 represents that a second event does not occur to the i^{th} patient.

### III. Data Generation Module

For the few-shot general disease set R, a data generation model is constructed based on the generative adversarial network of causal check, and the simulated data is generated by using the trained data generation model.

Specifically, the data generation model includes a generator and a discriminator. The generator G(z, c) is composed of multiple layers of generator modules, where z represents a random noise and c represents a disease label of a sample to be generated. The generator module includes a normalization layer, a fully-connected layer and an activation layer. The activation layer of the generator module of the last layer is a sigmoid activation layer, and the activation layers of other generator modules can be relu activation layer, sigmoid activation layer and tan h activation layer. The discriminator D is composed of multiple layers of discriminator modules, and the discriminator module includes a fully-connected layer, a Dropout layer and an activation layer.

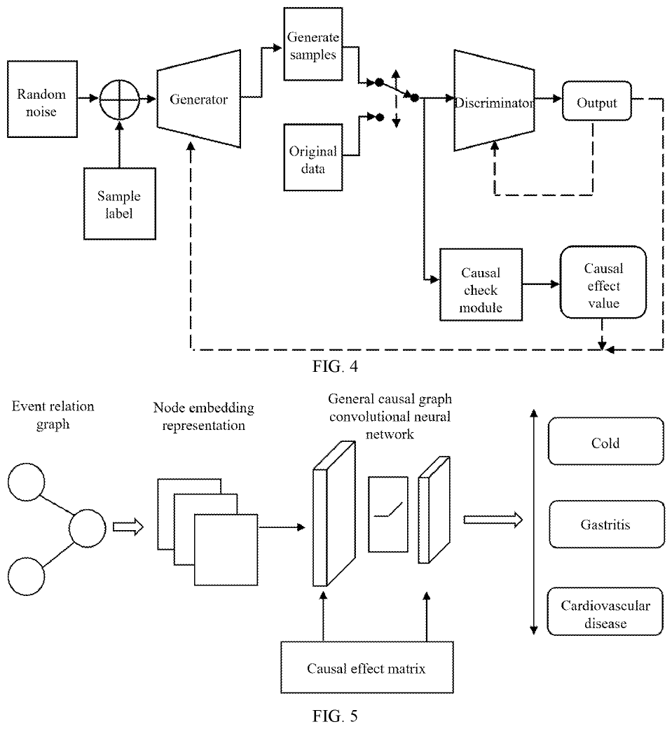

FIG. 4 is a structural diagram of a generative adversarial network based on causal check. The generator and discriminator are trained iteratively and alternately according to the training process of the generator and the discriminator, and finally the trained data generation model is obtained. The training process is described in detail below.

### (1) Training Process of the Generator

S1, for each disease r in the few-shot general disease set R, m_{g} noise points z^{r}={z^{1,r}, z^{2,r}, . . . , z^{m}^{_{g}}^{,r}} are randomly generated from the binomial distribution, and the corresponding disease label is c^{r}={r, r, . . . , r}. For all v diseases, N=m_{g}×v random noise data and disease label data are generated, the random noise data being z={z¹, z², . . . , z^{v}} and the disease label data being c={c¹, c², . . . , c^{v}}.

S2, the random noise z and the corresponding disease label c are input into the normalization layer of the first generator module, where the normalization layer is used for normalizing the input data, including batch standardization, sample standardization and the like; the normalized data are input into the fully connected layer of the first generator module to obtain a first characteristic representation of the input data; the first characteristic representation is input into the activation layer of the first generator module to obtain a second characteristic representation of the input data, and the second characteristic representation is input and output as the input data of the next generator module layer by layer; finally, the generated samples are obtained through the sigmoid activation layer of the generator module of the last layer.

S3, a causal check module is used to calculate the causal effect values of all event pairs of the generated samples.

S4, the generated samples and the disease labels are input into the discriminator, and the probability y* that the discriminator discriminates the generated samples as real data corresponding to the disease.

S5, the total loss L of the generator is calculated, including the adversarial loss L_{ζ} of the discriminator, a causal loss L_{causal} and a regularization term loss L_{regular}.

The adversarial loss of the discriminator measures the degree to which the generated sample of the generator is judged to be true by the discriminator. The smaller the adversarial loss of the discriminator, the easier it is for the generated sample to be judged to be true. The formula for calculating the adversarial loss L_{ζ} of the discriminator is as follows:

$${L}_{ζ} = \frac{1}{N} {\sum }_{i = 1}^{N} - log ( {y}_{i}^{*} )$$

where yᵢ* is the probability that the i^{th} generated sample is judged as the real data of the corresponding disease by the discriminator.

The causal loss measures the degree of causality between the generated sample of the generator and the original data. The smaller the causal loss, the more consistent the internal causality of the generated samples is with the original data. Specifically, the causal loss is a KL divergence loss between the causal effect values of all event pairs of the generated sample that are corrected by the frequency of the few-shot general disease and the causal effect values of all event pairs of original data. For a few-shot disease, the variance of the causal effect value corresponding to the calculated original data is large, and the stability of training is improved by giving a smaller weight. The calculation formula of the causal loss is as follows:

$${L}_{causal} = {\sum }_{r ∈ R} {q}_{r} {\sum }_{a ∈ {A}_{r}} ( {ATE}_{a , r}^{g} log ( {ATE}_{a , r}^{g} ) - {ATE}_{a , r}^{g} log ( {ATE}_{a , r}^{o} ) )$$

where, ATE_{a,r}^{o} represents a causal effect value of the first event variable a and a second event variable r of the original data, and ATE_{a,r}^{g} represents a causal effect value of the first event variable a and the second event variable r of the generated sample; Aᵣ represents the first event variable set paired with the second event variable r; qᵣ represents the frequency of a few-shot general disease r.

The calculation formula of the regularization term loss L_{regular} is as follows:

$${L}_{regular} = ‖ w ‖$$

where, ∥·∥ represents a norm L1 and w represents a parameter of the generator model.

The total loss of the generator is as follows:

$$L = {L}_{ζ} + {L}_{c a u s a l} + {L}_{r e g u l a r}$$

### (2) Training Process of the Discriminator

S1, m_{d} patient sample {(x₁, y₁), (x₂,y₂), . . . , (xₖ, yₖ), . . . , (xₘ_{_{d}}, yₘ_{_{d}})} are randomly extracted as positive samples from the original data, i.e., the general data set, where xₖ, yₖ respectively indicate the characteristic data and disease label of the extracted k^{th} positive sample.

S2, m_{d} patient samples {(x₁\hat{ }, y₁\hat{ }), (x₂\hat{ }, y₂\hat{ }), . . . , (xₖ\hat{ },yₖ\hat{ }), . . . , (xₘ_{_{d}}\hat{ },yₘ_{_{d}}\hat{ })} are randomly extracted from the original data as negative samples, where xₖ\hat{ },yₖ\hat{ } respectively 10 represent the characteristic data and disease label of the k^{th} negative sample. When sampling, it is necessary to ensure that the k^{th} positive sample and the k^{th} negative sample have different disease labels, that is, yₖ!=yₖ\hat{ }.

S3, m_{d} noise points {z₁\hat{ }, z₂\hat{ }, . . . , zₖ\hat{ }, . . . , zₘ_{_{d}}\hat{ }} are randomly sampled from the binomial distribution, and the generator is used to obtain generated samples, where the kl generated sample dₖ is expressed as follows:

$${d}_{k} = G ( \hat{{z}_{k}} , {y}_{k} )$$

S4, the extracted positive and negative samples and the generated samples are respectively input into the discriminator D to obtain the predicted disease label.

S5, the total loss L_{d} of the discriminator is calculated with the following formula:

$${L}_{d} = - \frac{1}{{m}_{d}} {\sum }_{k = 1}^{{m}_{d}} ( log ( D ( {x}_{k} , {y}_{k} ) ) + log ( 1 - D ( {x}_{k}^{∧} , {y}_{k} ) ) + log ( 1 - D ( {d}_{k} , {y}_{k} ) ) )$$

where D(xₖ, yₖ), D(xₖ\hat{ },yₖ), D(dₖ, yₖ) are respectively the probabilities that the positive sample, the negative sample and the generated sample are judged as the real data of the disease yₖ by the discriminator D.

IV. Model Prediction Module, the Implementation Process of which is Shown in FIG. 5.

The characteristic data and disease label data of the general patient to be trained are obtained. For diseases with insufficient training samples, the data generation model trained in the data generation module is used to generate general disease data. The training samples together with the generated general disease data are used to train the general multi-disease prediction model. The specific process is as follows:

Firstly, an event diagram is constructed, which is specifically as follows:

each first event variable in the first event variable set constitutes a first event node in the event relation graph, and each second event variable in the second event variable set constitutes a second event node in the event relation graph; an edge is constructed for each pair of the first event variable and the second event variable for each patient, thus completing the construction of the event relation graph.

Taking the first event variable set {fever, chest tightness} and the second event variable set {acute respiratory infection} of a patent as an example, an edge is constructed between fever and acute respiratory infection, and an edge is constructed between chest tightness and acute respiratory infection.

A graph representation learning algorithm is used to generate the embedding representations of the first event node and the second event node. Based on the event relation graph, the corresponding degree matrix Φ and adjacency matrix A are constructed. A causal effect matrix Ψ is constructed by using the causal effect values of the original data, and the numbers of rows and columns of the causal effect matrix Ψ are the same, which is the number of the first event nodes plus the number of the second event nodes. The element in row α and column β of the causal effect matrix Ψ is recorded as ψ_{α,β}, if row α is the first event node and column β is the second event node, ψ_{α,β}=ATE_{α,β}^{o} otherwise ψ_{α,β}=0.

A general multi-disease prediction model based on a general causal graph convolutional neural network is constructed. The general causal graph convolutional neural network includes several causal graph convolutional modules, and the causal graph convolutional module include a causal graph convolutional layer and an activation layer. The causal graph convolutional layer is a convolutional layer corrected by the causal effect matrix, and the robustness of the model is improved by adding causal effect correction. The embedding representation nodes are input into the causal graph convolutional layer of a first causal graph convolutional module to obtain a first graph characteristic representation h⁽⁰⁾;

$${h}^{( 0 )} = {Φ}^{- \frac{1}{2}} ( {A}^{∧} * Ψ ) {Φ}^{- \frac{1}{2}} {H}^{( 0 )} {W}^{( 0 )} \\ {A}^{∧} = A + I$$

where, H⁽⁰⁾ represents the node embedding representation, W⁽⁰⁾ represents the weight of the causal graph convolutional layer of the first causal graph convolutional module, which can be obtained by training; I represents an identity matrix, and * represents the multiplication of the elements of the matrix.

The first graph characteristic representation h⁽⁰⁾ is input into the activation layer of the first causal graph convolutional module to obtain an output H⁽¹⁾ of the first causal graph convolutional module;

$${H}^{( 1 )} = σ ( {h}^{( 0 )} )$$

Where σ(·) represents an activation function.

The output of the previous causal graph convolutional module is input into the next causal graph convolutional module until the final disease prediction result is obtained. The loss of the general causal graph convolutional neural network is calculated, and the loss function is a cross entropy loss function.

The general causal graph convolutional neural network is iteratively trained to obtain the trained general multi-disease prediction model, and the trained general multi-disease prediction model is used to predict general diseases.

Aiming at the general scene, the present application provides a general propensity score network suitable for calculating the general propensity scores; a causal effect calculation method is used to perform causal check for the general data generated by the generative adversarial network, so that the generated data is more in line with the real causal logic; in the training process of the generator, the same number of noise points are generated from binomial distribution for each few-shot disease to serve as the input of the generator together; in the training process of the discriminator, positive samples are extracted from the original data, and the same number of samples with different labels are extracted as negative samples, which are used to train the discriminator together with the negative samples generated by the generator; aiming at the few-shot general diseases, the generative adversarial network based on causal check is used to amplify the general data, so as to improve the prediction performance of the general multi-disease prediction system for the few-shot diseases; a general multi-disease prediction model based on a general causal graph convolutional neural network is proposed, and the causal effect value is integrated to improve the prediction performance of the general multi-disease prediction system.

It should also be noted that the terms “including”, “comprising” or any other variation thereof are intended to cover non-exclusive inclusion, so that a process, method, commodity or equipment including a series of elements includes not only those elements, but also other elements not explicitly listed, or elements inherent to such process, method, commodity or equipment. Without more restrictions, an element defined by the phrase “including a” does not exclude the existence of other identical elements in the process, method, commodity or equipment including the element.

Specific embodiments of this specification have been described above. Other embodiments shall also be within the scope of the appended claims. In some cases, the actions or steps recited in the claims may be performed in a different order than in the embodiments and still achieve the desired results. In addition, the processes depicted in the drawings do not necessarily require the specific order shown or the sequential order to achieve the desired results. In some embodiments, multitasking and parallel processing are also possible or may be advantageous.

The above is only the preferred embodiments of one or more embodiments of this specification, and it is not intended to limit one or more embodiments of this specification. Any modification, equivalent substitution, improvement and the like made within the spirit and principle of one or more embodiments of this specification shall be included in the scope of protection of one or more embodiments of this specification.

What is claimed is:

1. A general multi-disease prediction system based on causal check data generation, comprising:
(1) a disease statistics module configured to count a sample number of various general diseases, and obtain few-shot general diseases according to a sample ratio of various general diseases; wherein the sample ratio is a ratio of a sample number of diseases with a largest number of samples to the sample number of various general diseases, and for a general disease with a sample ratio greater than a set threshold, the general disease is added into a few-shot general disease set R, and a frequency of a r^{th} few-shot general disease is calculated,
$${q}_{tr} = \frac{{count}_{r}}{{Σ}_{r ∈ R} {count}_{r}} ,$$
where countᵣ is a sample number of the r^{th} few-shot general disease;
(2) a causal check module configured to construct a first event variable set according to a characteristic variable set of a general patient and construct a second event variable set according to a disease label variable set of the general patient, wherein an event pair is formed by any first event variable and any second event variable;
wherein the causal check module is further configured to construct and train a general propensity score network and calculate a general propensity score by using a trained general propensity score network, wherein a general propensity score represents a probability that a first event occurs to the general patient under a covariant condition; and causal effect values of all event pairs are calculated by using the general propensity score;
(3) a data generation module configured to construct a data generation model for the few-shot general diseases based on a generative adversarial network of causal check, and generate simulated data by using a trained data generation model;
wherein the data generation model comprises a generator and a discriminator, and the generator and the discriminator are trained iteratively and alternately;
a training process of the generator comprises: generating a random noise for each few-shot general disease, and inputting the random noise and a corresponding disease label into the generator to obtain a generated sample; calculating the causal effect values of all event pairs of the generated sample; inputting the generated sample and the corresponding disease label into the discriminator to obtain a discrimination result; wherein a total loss of the generator comprises a discriminator adversarial loss, a causal loss and a regularization term loss; the causal loss is a KL divergence loss between the causal effect values of all event pairs of the generated sample, corrected by a frequency of the few-shot general disease, and the causal effect values of all event pairs of original data;
a training process of the discriminator comprises: randomly extracting positive samples from the original data, and extracting negative samples with a same number with the positive samples but with different disease labels; generating a same number of random noises, and using the generator to obtain the generated sample; inputting the positive samples, the negative samples and the generated sample into the discriminator, respectively, to obtain the discrimination result; and
(4) a model prediction module configured to obtain characteristic data and disease label data of a general patient to be trained, and generate general disease data by using the data generation model for the few-shot general diseases; wherein training samples and generated general disease data are jointly used to train a general multi-disease prediction model based on a general causal graph convolutional neural network, and a trained general multi-disease prediction model is used to predict general diseases.

2. The system according to claim 1, wherein in the causal check module, the general propensity score network is trained by using binary variable data of the general patient; characteristic variable data and label variable data of the general patient are converted into binary variables, categorical variables are converted into the binary variables by One-Hot Encoding, and continuous variables are converted into the categorical variables by binning in advance, and the categorical variables are further converted into the binary variables by One-Hot Encoding.

3. The system according to claim 1, wherein the general propensity score network comprises an input layer, a locally-connected layer, a sigmoid activation layer and an output layer;
a number of codes in the input layer and a number of codes in the output layer are both a number M of first event variables in the first event variable set; both the locally-connected layer and the sigmoid activation layer comprise τM nodes, τ≥2; a u^{th} node of the input layer is connected with all nodes except those from a τ(u−1)+1^{th} to a τu^{th} node in the locally-connected layer; the nodes from the τ(u−1)+1^{th} to the τu^{th} node in the locally-connected layer are connected with nodes from a τ(u−1)+1^{th} to a τu^{th} node in the sigmoid activation layer in one-to-one correspondence; the nods from the τ(u−1)+1^{th} to the τu^{th} node in the sigmoid activation layer are only connected with a u^{th} node in the output layer.

4. The system according to claim 3, wherein the training process of the general propensity score network is as follows:
for each first event variable a, covariant data corresponding to the training samples is input into the locally-connected layer to obtain a first characteristic representation of propensity; the first characteristic representation of propensity is input into the sigmoid activation layer to obtain a second characteristic representation of propensity; the second characteristic representation of propensity is input into the output layer to obtain a predicted value of the first event variable a; a propensity loss is calculated by using the predicted values of all the first event variables and true values of all the first event variables.

5. The system according to claim 1, wherein in the causal check module, the trained general propensity score network is used to calculate a general propensity score pᵢ^{a} of a general patient i for a first event variable a, and a causal effect value ATE_{a,b} of the first event variable a and a second event variable b are calculated by using the general propensity score according to the following calculation formula:
$${ATE}_{a , b} = \frac{1}{n} {Σ}_{i = 1}^{N} \frac{{T}_{i} {Y}_{i}}{{p}_{i}^{a}} - \frac{1}{N} {Σ}_{i = 1}^{n} \frac{( 1 - {T}_{i} ) {Y}_{i}}{1 - {p}_{i}^{a}}$$
where n represents a total number of patients to be studied, Tᵢ represents a true value of the first event variable of an i^{th} patient, and Yᵢ represents a true value of the second event variable of the i^{th} patient.

6. The system according to claim 1, wherein in the data generation module, the generator is composed of multiple layers of generator modules; the generator module comprises a normalization layer, a fully-connected layer and an activation layer; the activation layer of the generator module in a last layer is a sigmoid activation layer; in a training process, the random noises and the corresponding disease label are input into the normalization layer of a first generator module, and normalized data are input into the fully connected layer of the first generator module to obtain a first characteristic representation of input data; the first characteristic representation is input into the activation layer of the first generator module to obtain a second characteristic representation of the input data, the second characteristic representation is used as input data of the generator module in a next layer, and the generated sample is obtained through the sigmoid activation layer of the generator module in the last layer.

7. The system according to claim 1, wherein in the data generation module, a formula for calculating a causal loss L_{causal} is as follows:
$${L}_{casual} = {\sum }_{r ∈ R} {q}_{r} {\sum }_{a ∈ {A}_{r}} ( {ATE}_{a , r}^{g} log ( {ATE}_{a , r}^{g} ) - {ATE}_{a , r}^{g} log ( {ATE}_{a , r}^{o} ) )$$
where ATE_{a,r}^{o} represents a causal effect value of a first event variable a and a second event variable r of the original data, and ATE_{a,r}^{o} represents a causal effect value of the first event variable a and the second event variable r of the generated sample; Aᵣ represents the first event variable set paired with the second event variable r; the second event variable set is a general disease set, and the second event variable r corresponds to a few-shot general disease r in the few-shot general disease set R.

8. The system according to claim 1, wherein in the data generation module, a formula for calculating a discriminator adversarial loss L_{ζ} is as follows:
$${L}_{ζ} = \frac{1}{N} {\sum }_{i = 1}^{N} - log ( {y}_{i}^{*} )$$
where N is a data size of the random noises, and yᵢ* is a probability that an i^{th} generated sample is determined as real data of a corresponding disease by the discriminator;
a calculation formula of a regularization term loss L_{regular} is as follows:
$${L}_{regular} = ‖ w ‖$$
where ∥·∥ represents a L1 norm, and w represents a model parameter of the generator.

9. The system according to claim 1, wherein in the data generation module, a formula for calculating a total loss L_{d} of the discriminator is as follows:
$${L}_{d} = - \frac{1}{{m}_{d}} {\sum }_{k = 1}^{{m}_{d}} ( log ( D ( {x}_{k} , {y}_{k} ) ) + log ( 1 - D ( {x}_{k}^{∧} , {y}_{k} ) ) + log ( 1 - D ( {d}_{k} , {y}_{k} ) ) )$$
where is m_{d} is a number of the positive samples, yₖ is a disease label corresponding to the positive samples, and xₖ, xₖ\hat{ }, dₖ are k^{th} extracted positive sample, k^{th} extracted negative sample and k^{th} generated sample obtained by using the generator, respectively, and D(xₖ, yₖ), D(xₖ, yₖ), D(dₖ, yₖ) are probabilities that the positive sample xₖ, the negative sample xₖ\hat{ } and the generated sample dₖ are determined as the real data of a disease yₖ by the discriminator.

10. The system according to claim 1, wherein the model prediction module is configured to:
construct an event relation graph: each first event variable constitutes a first event node in the event relation graph, each second event variable constitutes a second event node in the event relation graph, and an edge is constructed for each event pair;
generate a node embedding representation of the first event node and a node embedding representation of the second event node, construct a degree matrix Φ and an adjacency matrix A based on the event relation graph, and construct a causal effect matrix Ψ using the causal effect values of the original data;
construct the general multi-disease prediction model based on the general causal graph convolutional neural network, wherein the general causal graph convolutional neural network comprises a plurality of causal graph convolutional modules, and each of the causal graph convolutional module comprises a causal graph convolutional layer and an activation layer;
input the node embedding representations into the causal graph convolutional layer of a first causal graph convolutional module to obtain a first graph characteristic representation h⁽⁰⁾:
$${h}^{( 0 )} = {{Φ}^{- \frac{1}{2}} ( ( A + I ) * Ψ )}^{- \frac{1}{2}} {H}^{( 0 )} {W}^{( 0 )}$$
where H⁽⁰⁾ represents a node embedding representation, W⁽⁰⁾ represents a weight of the causal graph convolutional layer, I represents an identity matrix, and * represents a multiplication of elements of the matrix;
input h⁽⁰⁾ into the activation layer of the first causal graph convolutional module to obtain an output H⁽¹⁾ of the first causal graph convolutional module; and
input the output of a previous causal graph convolutional module into a next causal graph convolutional module until a final disease prediction result is obtained.
